(19) United States
(12) Patent Application Publication
(10) Pub. No.: US 2024/0218089 A1
(43) Pub. Date: Jul. 4, 2024

(54) OLIGOMERIZATION OF ALPHA OLEFINS USING SUPPORTED METALLOCENE CATALYSTS IN SELECTIVE PRODUCTION OF VINYLIDENE DIMERS

(71) Applicant: Chevron Phillips Chemical Company LP, The Woodlands, TX (US)

(72) Inventors: Graham R. Lief, Bartlesville, OK (US); Thomas J. Malinski, Porter, TX (US); Qing Yang, Bartlesville, OK (US); Steven M. Bischof, Spring, TX (US); Eric Haschke, Bartlesville, OK (US)

(21) Appl. No.: 18/534,805

(22) Filed: Dec. 11, 2023

Related U.S. Application Data
(60) Provisional application No. 63/387,515, filed on Dec. 15, 2022.

(51) Int. Cl. C08F 4/76; C08F 4/52; C08F 110/14

(52) U.S. Cl. CPC C08F 4/76; C08F 4/52; C08F 110/14; C08F 2410/04; C08F 2420/10

(57) ABSTRACT

The present disclosure generally relates to processes for the oligomerization of alpha olefins with high yield to dimer and trimer oligomer products, where heavier products including tetramers are relatively minimal. These processes utilize catalyst compositions comprising a metallocene containing at least one indenyl ligand, and the indenyl ligand can have at least one halogenated substituent, such as a fluorinated substituent. Such oligomerization processes utilizing the disclosed metallocene-based catalyst systems demonstrate increased olefin conversion without an accompanying shift of the product distribution toward heavier products.

### REFERENCE TO RELATED APPLICATION

This application claims the benefit of U.S. Provisional Patent Application No. 63/387,515, filed on Dec. 15, 2022, the disclosure of which is incorporated herein by reference in its entirety.

### FIELD OF THE INVENTION

The present disclosure generally relates to processes for the oligomerization of alpha olefins to oligomer products having high vinylidene content using metallocene catalyst systems, and more particularly, relates to the production of low volatility and low viscosity polyalphaolefins for use in lubricant formulations and other related end-use applications.

### BACKGROUND OF THE INVENTION

It can be advantageous for processes for the oligomerization of alpha olefins to produce a relatively light product mixture comprising a high content of dimers and trimers, while also limiting the amount of larger molecular weight oligomers formed. In addition, alpha olefin dimers can be produced as an internal olefin, a branched olefin, or a vinylidene olefin having a terminal carbon-carbon double bond. Of these, vinylidene dimers often are preferred for their relatively high reactivity, as compared to internal and branched dimers. Therefore, oligomerization processes that provide high conversion of the alpha olefin reactants to light molecular weight product mixtures with high vinylidene dimer and trimer content are desirable. Accordingly, it is to these ends that the present disclosure is generally directed.

### SUMMARY OF THE INVENTION

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Processes disclosed herein can comprise contacting a catalyst composition with a C₄ to C₃₀ alpha olefin monomer and optionally H₂ under oligomerization conditions to produce an oligomer product comprising at least 50 mol % alpha olefin dimer. In certain aspects, the catalyst composition can comprise a metallocene compound, a chemically treated solid oxide, and a co-catalyst. In these aspects, the metallocene compound can have one of the following formulas:

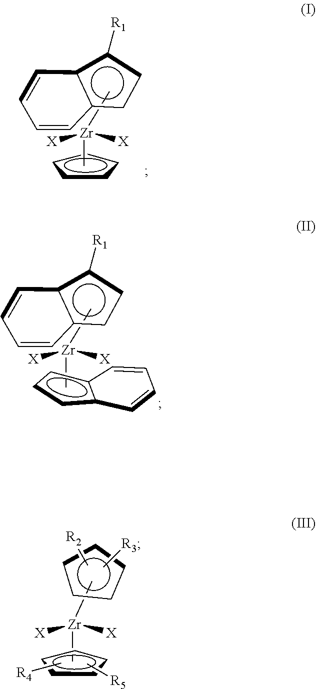

wherein R₁ is a C₁-C₂₀ hydrocarbyl group or a halogen-substituted C₁-C₂₀ hydrocarbyl group, each X independently is a halogen or a C₁-C₁₈ hydrocarbyl group, and R₂, R₃, R₄, R₅ independently are H or a C₁-C₁₈ hydrocarbyl group.

In other aspects, the catalyst composition can comprise a metallocene compound, an activator, and an optional co-catalyst, wherein the activator comprises an aluminoxane compound, an organoboron or organoborate compound, an ionizing ionic compound, a chemically treated solid oxide, or a combination thereof. In such aspects, the metallocene compound can have one of the following formulas:

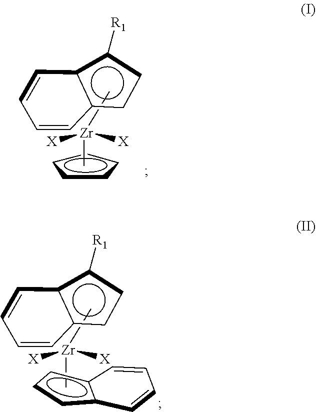

wherein R₁ is a halogen-substituted C₁-C₂₀ hydrocarbyl group, and each X independently is a halogen or a C₁-C₁₈ hydrocarbyl group. In a particular aspect, the activator comprises a chemically treated solid oxide and the catalyst composition is substantially free of aluminoxane compounds, organoboron or organoborate compounds, ionizing ionic compounds, or combinations thereof.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the examples and detailed description.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the systems, processes, or methods described herein are contemplated with or without explicit description of the particular combination. Additionally, unless explicitly recited otherwise, any aspect or feature disclosed herein can be combined to describe inventive systems, processes, or methods consistent with the present disclosure.

In this disclosure, while systems and processes are described in terms of “comprising” various components or steps, the systems and processes also can “consist essentially of” or “consist of” the various components or steps, unless stated otherwise.

The terms “a,” “an,” and “the” are intended to include plural alternatives, e.g., at least one. For instance, the disclosure of “a catalyst” is meant to encompass one catalyst, or mixtures or combinations of more than one catalyst, unless otherwise specified.

The terms “contacting” and “combining” are used herein to describe compositions, processes/methods, and systems in which the materials are contacted or combined together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique. Herein, “contacting” or “combining” two or more components can result in a reaction product.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, transition metals for Group 3-12 elements, and halogens for Group 17 elements.

The term “hydrocarbon” refers to a compound containing only carbon and hydrogen. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon). The term “alkane” refers to a saturated hydrocarbon compound.

The term “olefin” refers to hydrocarbons that have at least one carbon-carbon double bond that is not part of an aromatic ring or an aromatic ring system. The term “olefin” includes aliphatic and aromatic, cyclic and acyclic, and/or linear and branched hydrocarbons having at least one carbon-carbon double bond that is not part of an aromatic ring or ring system unless specifically stated otherwise. Olefins having only one, only two, only three, etc., carbon-carbon double bonds can be identified by use of the term “mono,” “di,” “tri,” etc., within the name of the olefin. The olefins can be further identified by the position of the carbon-carbon double bond(s).

The term “alpha olefin” as used herein refers to any olefin that has 1) a carbon-carbon double bond between the first and second carbon atom of the longest contiguous chain of carbon atoms, and 2) at least one hydrogen atom bound to the second carbon of the chain. The term “alpha olefin” includes linear and branched alpha olefins and alpha olefins which can have more than one non-aromatic carbon-carbon double bond, unless expressly stated otherwise. In the case of branched olefins, a branch can be at the 2-position of a 1-alkene (a vinylidene) with respect to the olefin double bond. By itself, the term “alpha olefin” does not indicate the presence or absence of heteroatoms and/or the presence or absence of other carbon-carbon double bonds unless explicitly indicated. The terms “hydrocarbon alpha olefin” or “alpha olefin hydrocarbon” refer to alpha olefin compounds containing only hydrogen and carbon.

The term “normal alpha olefin” refers to the general structure H₂C═CRH. The term “vinylidene” refers to the general structure H₂C═CRR′. The term “internal olefin” refers to the general structure RHC═CR′H. Finally, the term “trisubstituted olefin” refers to the general structure RHC═CR′R″. In each case, R, R′, and R″ may be the same or different.

The terms “oligomerization” and “oligomerizing” refer to processes which produce an oligomer product comprising at least 20 wt. %, 35 wt. %, 50 wt. %, or 60 wt. % products comprising from 2 to 20 monomer units, including dimers, trimers, tetramers, and so forth. Thus, oligomer refers to a compound that contains from 2 to 20 monomer units, including dimers, trimers, tetramers, and so forth. The terms “oligomerization product” and “oligomer product” include all products made by the “oligomerization” process, including the “oligomers” and products which are not “oligomers” (e.g., products which contain more than 20 monomer units, or solid polymer), but exclude other potential non-oligomer components of an oligomerization reactor effluent stream, such as unreacted monomer, catalyst, solvent, and hydrogen, amongst other components. It should be noted that the monomer units in the “oligomer” or “oligomer product” do not have to be the same. For example, these terms are also used generically herein to include olefin homo-oligomers, co-oligomers, and so forth, and thus encompass products derived from any number of different olefin monomers disclosed herein.

For any particular compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

Features within this disclosure that are provided as minimum values can be alternatively stated as “at least” or “greater than or equal to” any recited minimum value for the feature disclosed herein. Features within this disclosure that are provided as maximum values can be alternatively stated as “less than or equal to” or “below” any recited maximum value for the feature disclosed herein.

Several types of ranges are disclosed herein. When a range of any type is disclosed or claimed, the intent is to disclose or claim individually each possible number that such a range could reasonably encompass, including end points of the range as well as any sub-ranges and combinations of sub-ranges encompassed therein. For example, the weight ratio of the metallocene compound to the chemically treated solid oxide in the catalyst composition can be in various ranges. By a disclosure that the weight ratio of the metallocene compound to the chemically treated solid oxide can range from 1:10 to 1:10,000, the intent is to recite that the weight ratio can be any ratio within the range and, for example, can include any range or combination of ranges from 1:10 to 1:10,000, such as from 1:10 to 1:1,000, from 1:10 to 500:1, or from 1:10 to 1:100, and so forth. Likewise, all other ranges disclosed herein should be interpreted in a manner similar to this example.

In general, an amount, size, formulation, parameter, range, or other quantity or characteristic is “about” or “approximate,” whether or not it is expressly stated to be such. Whether or not modified by the term “about” or “approximately,” the claims include equivalents to the quantities or characteristics.

Although any methods, devices, and materials similar or equivalent to those described herein can be used in the practice or testing of the disclosure, the typical methods, devices, and materials are herein described.

All publications and patents mentioned herein are incorporated by reference in their entirety for the purpose of describing and disclosing, for example, the constructs and methodologies that are described in the publications and patents, which might be used in connection with the present disclosure.

### DETAILED DESCRIPTION OF THE INVENTION

Disclosed herein are processes for the oligomerization of alpha olefins using metallocene-based catalyst compositions. These processes can result in an unexpectedly high relative amount of trimers and dimers of the alpha olefin, particularly vinylidene dimers. The alpha olefin conversion in oligomerization processes employing heterogenous metallocene catalyst compositions comprising chemically treated solid oxides can be improved by conducting the process in the presence of H₂, typically accompanied by a shift in the product mixture toward heavier, and more stable (i.e., less reactive) oligomer products. Surprisingly, the oligomerization processes using H₂ did not shift the product distribution to a heavier oligomer distribution, as compared to otherwise identical homogenous catalyst compositions lacking a chemically treated solid oxide.

### Catalyst Compositions

Processes disclosed herein can comprise contacting a catalyst composition with an alpha olefin monomer under oligomerization conditions to product an oligomer product. In certain aspects, the catalyst composition can comprise a metallocene compound, a chemically treated solid oxide, and a co-catalyst. In other aspects, the catalyst composition can comprise a metallocene compound, an activator, and an optional co-catalyst. The activator can comprise an aluminoxane compound, an organoboron or organoborate compound, an ionizing ionic compound, a chemically treated solid oxide, or a combination thereof. When the activator in the catalyst composition is a chemically treated solid oxide (activator), then aluminoxane, organoboron or organoborate, and ionizing ionic materials, if present, are referred to as co-catalysts. One or more than one metallocene compound, activator, or co-catalyst can be present in the catalyst composition.

In circumstances where the catalyst composition contains a metallocene compound, a chemically treated solid oxide, and an organoaluminum co-catalyst, the catalyst composition can be substantially free of aluminoxanes, organoboron or organoborate compounds, ionizing ionic compounds, and/or other similar materials; alternatively, substantially free of aluminoxanes; alternatively, substantially free or organoboron or organoborate compounds; or alternatively, substantially free of ionizing ionic compounds. In these aspects, the catalyst composition has catalyst activity, discussed herein, in the absence of these additional materials. For example, a catalyst composition of the present invention can consist essentially of the metallocene compound, the chemically treated solid oxide, and the organoaluminum co-catalyst, wherein no other materials are present in the catalyst composition which would increase/decrease the activity of the catalyst composition by more than about 10% from the catalyst activity of the catalyst composition in the absence of said materials.

Generally, the metallocene compound in the catalyst composition can be any that are described in U.S. Pat. No. 11,186,665. For example, the metallocene compound can have the formula:

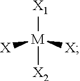

wherein M can be titanium, hafnium, or zirconium, or alternatively zirconium; X¹ can be a substituted cyclopentadienyl or indenyl ligand wherein at least one substituent (R¹) is a halogen-substituted C₁-C₂₀ hydrocarbyl group; X² can be a substituted or unsubstituted cyclopentadienyl ligand or a substituted or unsubstituted indenyl ligand; wherein X¹ and X² are unbridged; and each X is independently selected from a halide, hydride, a C₁-C₂₀ hydrocarbyl group, a C₁-C₂₀ heterohydrocarbyl group, tetrahydroborate, or OBR^{A}₂ or OSO₂R^{A}, wherein each R^{A} independently is a C₁-C₁₂ hydrocarbyl group.

According to a further aspect, the groups X of the metallocene compound each can be independently selected from F, Cl, Br, a C₁-C₁₂ hydrocarbyloxide group, a C₁-C₁₂ hydrocarbylamino group, or a trihydrocarbylsilyl group, wherein each hydrocarbyl is independently a C₁-C₁₂ hydrocarbyl group. In other aspects, each X can be Cl.

Certain metallocene compounds demonstrate advantageous properties when employed within the oligomerization processes contemplated herein. Particularly, metallocene compounds of the following formulas can demonstrate an alpha olefin conversion and favorable oligomer product distribution with respect to the amount of the dimer (e.g., vinylidene dimer) and trimer products formed within the product mixture when such metallocenes are present in the catalyst composition.

For instance, the metallocene compound of catalyst compositions disclosed herein can have one of the following formulas (I)-(III) as represented below:

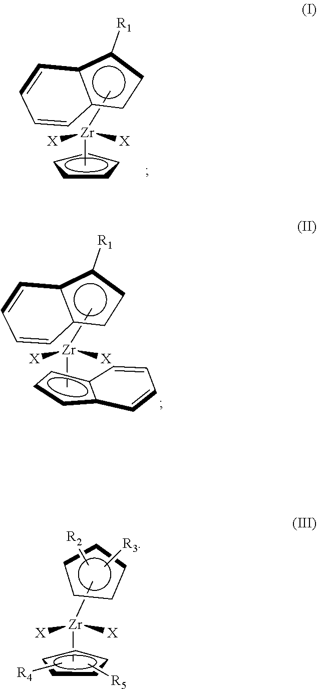

wherein R₁ is a C₁-C₂₀ hydrocarbyl group or a halogen-substituted C₁-C₂₀ hydrocarbyl group and each X independently is a halogen or a C₁-C₁₈ hydrocarbyl group. In aspects where the metallocene compound has Formula (C), R₂, R₃, R₄, R₅ independently can be H, a C₁-C₁₈ hydrocarbyl group, a C₁-C₁₂ hydrocarbyl group, a C₁-C₈ hydrocarbyl group, or a C₁-C₆ hydrocarbyl group.

The halogen-substituted hydrocarbyl substituent of R¹ can be selected from a C₁-C₂₀ hydrocarbyl group substituted with one or more fluoro-, chloro-, bromo-, or iodo-substituents, or a combination thereof, independently selected. In some aspects, the halogen-substituted hydrocarbyl substituent of R¹ is a C₁-C₂₀ hydrocarbyl group or a C₁-C₁₂ hydrocarbyl group substituted with one or more fluoro-, chloro-, or bromo-substituents. In an aspect, R¹ can be a halogen-substituted C₁-C₂₀ hydrocarbyl group comprising 1, 2, 3, 4, 5, 6, 7, 8, or more halogen atoms such as fluorine atoms, including ranges between any of these numbers, as allowed by the size and structure of a particular hydrocarbyl group. For example, when the halogen-substituted C₁-C₂₀ hydrocarbyl group is a phenyl group, the upper limit of halogen substituents is five (5) substituents, and the phenyl group can include 1, 2, 3, 4, or 5 halogen substituents. In other aspects, the halogen-substituted C₁-C₂₀ hydrocarbyl group can comprise from 1 to 8, from 2 to 8, from 1 to 7, from 2 to 7, from 1 to 6, from 2 to 6, from 1 to 5, from 2 to 5, from 1 to 4, from 2 to 4, from 1 to 3, or from 2 to 3 halogen atoms.

For example, in one aspect, the halogen-substituted hydrocarbyl substituent of R¹ of the metallocene compound can be a C₁-C₂₀ aliphatic or C₆-C₂₀ aromatic group substituted with at least two fluoro-, chloro-, or bromo-substituents, or a combination thereof. In another aspect, the halogen-substituted hydrocarbyl substituent of R¹ of the metallocene compound can be selected from a fluoro-disubstituted, chloro-disubstituted, or bromo-disubstituted C₁-C₁₂ alkyl, C₂-C₁₂ alkenyl, C₃-C₇cycloalkyl, C₃-C₇cycloalkenyl, C₆-C₁₀ aryl, or C₇-C₁₂ aralkyl. In still a further aspect, the halogen-substituted hydrocarbyl substituent of R¹ of the metallocene compound can be further substituted with at least one additional substituent selected from a C₁-C₁₂ hydrocarbyl group.

Metallocene compounds are contemplated with R¹ as a halogenated phenyl group or a halogenated benzyl group. In certain aspects, R₁ can be a 2,6-diflourophenyl group, a 2,6-difluorobenzyl group, a 2,4,6-trifluorophenyl group, a 2,4,6-trifluorobenzyl group, a pentafluorophenyl group, or a pentafluorobenzyl group.

Exemplary metallocene compounds embodying the formulas and descriptions above are provided as metallocenes (A)-(F) as depicted below:

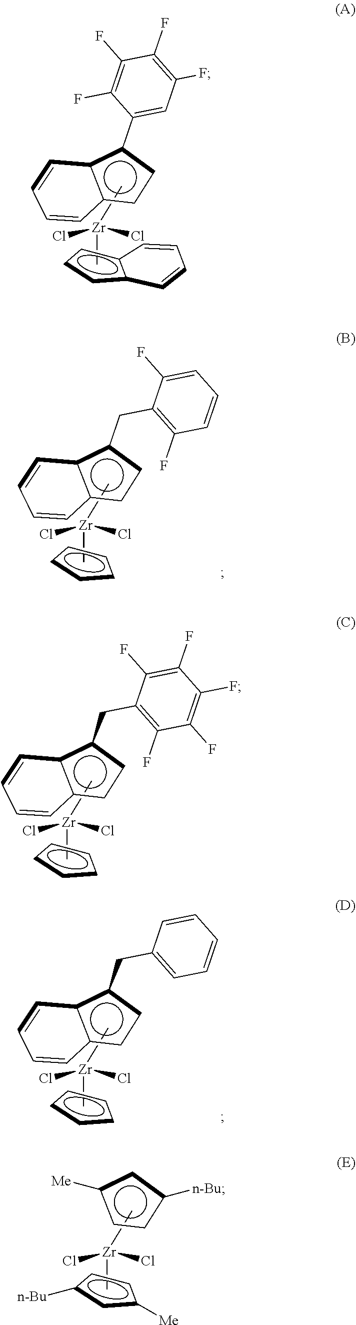

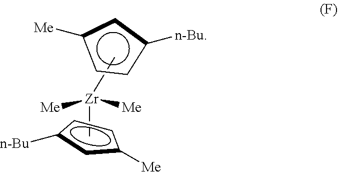

Catalyst compositions comprising each of metallocenes (A)-(F) were prepared as examples below as both heterogenous and homogenous catalysts incorporating various activators and co-catalysts. As discussed herein, the activator can comprise an aluminoxane compound, an organoboron or organoborate compound, an ionizing ionic compound, a chemically treated solid oxide, or a combination thereof.

Aluminoxanes that can serve as activators in this disclosure are generally represented by formulas such as (R¹²—Al—O)ₙ, R¹²(R¹²—Al—O)ₙAl(R¹²)₂, and the like, wherein the R¹² group is typically a linear or branched C₁-C₆ alkyl such as methyl, ethyl, propyl, butyl, pentyl, or hexyl wherein n typically represents an integer from 1 to 50. In one aspect, the aluminoxane compound used in the disclosed catalyst composition can include, but is not limited to, methylaluminoxane (MAO), ethylaluminoxane, modified methylaluminoxane (MMAO) such as an isobutyl-modified methyl alumoxane, n-propylaluminoxane, iso-propylaluminoxane, n-butylaluminoxane, t-butyl-aluminoxane, sec-butylaluminoxane, iso-butylaluminoxane, t-butyl aluminoxane, 1-pentyl-aluminoxane, 2-pentylaluminoxane, 3-pentylaluminoxane, iso-pentylaluminoxane, neopentylaluminoxane, or combinations thereof.

While aluminoxanes with different types of “R” groups such as R¹² are encompassed by the present disclosure, methyl aluminoxane (MAO), ethyl aluminoxane, or isobutyl aluminoxane are typical aluminoxane activators used in the catalyst compositions of this disclosure. These aluminoxanes are prepared from trimethylaluminum, triethylaluminum, or triisobutylaluminum, respectively, and are sometimes referred to as poly(methylaluminum oxide), poly(ethylaluminum oxide), and poly(isobutylaluminum oxide), respectively. It is also within the scope of the disclosure to use an aluminoxane in combination with a trialkylaluminum, such as disclosed in U.S. Pat. No. 4,794,096.

Organoboron compounds that can be used in the catalyst composition of this disclosure are similarly varied. In one aspect, the organoboron compound can comprise neutral boron compounds, borate salts, or combinations thereof. For example, the organoboron compounds of this disclosure can comprise a fluoroorgano boron compound, a fluoroorgano borate compound, or a combination thereof. Any fluoroorgano boron or fluoroorgano borate compound known in the art can be utilized. The term fluoroorgano boron compound has its usual meaning to refer to neutral compounds of the form BY₃. The term fluoroorgano borate compound also has its usual meaning to refer to the monoanionic salts of a fluoroorgano boron compound of the form [cation]+[BY₄]⁻, where Y represents a fluorinated organic group. For convenience, fluoroorgano boron and fluoroorgano borate compounds are typically referred to collectively by organoboron and organoborate compounds, or by either name as the context requires.

Examples of organoboron or organoborate compounds that can be used as activators in the present disclosure include, but are not limited to, fluorinated aryl borates such as, N,N-dimethylanilinium tetrakis(pentafluorophenyl)borate (DTPB), triphenylcarbenium tetrakis(pentafluorophenyl)borate, lithium tetrakis(pentafluorophenyl)borate, N,N-dimethylanilinium tetrakis[3,5-bis(trifluoro-methyl)phenyl]borate, triphenylcarbenium tetrakis[3,5-bis(trifluoromethyl)phenyl]borate, and the like, including mixtures thereof; alternatively, N,N-dimethylanilinium tetrakis-(pentafluorophenyl)borate (DTBP); alternatively, triphenylcarbenium tetrakis(pentafluorophenyl)borate; alternatively, lithium tetrakis(pentafluorophenyl)borate; alternatively, N,N-dimethylanilinium tetrakis[3,5-bis(trifluoromethyl)phenyl]borate; or alternatively, triphenylcarbenium tetrakis[3,5-bis(trifluoromethyl)phenyl]borate. Other suitable organoboron or organoborate activators include, but are not limited to, tris(pentafluorophenyl)-boron, tris[3,5-bis(trifluoromethyl)phenyl]boron, and the like, including mixtures thereof.

Although not intending to be bound by the following theory, these examples of organoboron and organoborate compounds, and related compounds, are thought to form “weakly-coordinating” anions when combined with organometal compounds, as disclosed in U.S. Pat. No. 5,919,983.

An ionizing ionic compound is an ionic compound which can function to enhance the activity of the catalyst composition. Examples of ionizing ionic compounds that may be suitable as activators in catalyst compositions disclosed herein include, but are not limited to, the following compounds: tri(n-butyl)ammonium tetrakis(p-tolyl)borate, tri(n-butyl) ammonium tetrakis(m-tolyl)borate, tri(n-butyl)ammonium tetrakis(2,4-dimethylphenyl)borate, tri(n-butyl)ammonium tetrakis(3,5-dimethylphenyl)borate, tri(n-butyl)ammonium tetrakis[3,5-bis(trifluoro-methyl)phenyl]borate, tri(n-butyl)ammonium tetrakis(pentafluorophenyl)borate, N,N-dimethylanilinium tetrakis(p-tolyl)borate, N,N-dimethylanilinium tetrakis(m-tolyl)borate, N,N-dimethylanilinium tetrakis(2,4-dimethylphenyl)borate, N,N-dimethylanilinium tetrakis(3,5-dimethylphenyl)borate, N,N-dimethylanilinium tetrakis[3,5-bis(trifluoromethyl)phenyl]borate, N,N-dimethylanilinium tetrakis(pentafluorophenyl)borate, triphenylcarbenium tetrakis(p-tolyl)borate, triphenylcarbenium tetrakis(m-tolyl)borate, triphenylcarbenium tetrakis(2,4-tetrakis(3,5-dimethylphenyl)borate, dimethylphenyl)borate, triphenylcarbenium triphenylcarbenium tetrakis[3,5-bis(trifluoromethyl)phenyl] borate, triphenylcarbenium tetrakis(pentafluorophenyl)borate, tropylium tetrakis(p-tolyl)borate, tropylium tetrakis(m-tolyl)borate, tropylium tetrakis(2,4-dimethylphenyl)borate, tropylium tetrakis(3,5-dimethylphenyl)borate, tropylium tetrakis[3,5-bis(trifluoromethyl)phenyl]borate, tropylium tetrakis(pentafluorophenyl) borate, lithium tetrakis(pentafluorophenyl)borate, lithium tetraphenylborate, lithium tetrakis(p-tolyl)borate, lithium tetrakis(m-tolyl)borate, lithium tetrakis(2,4-dimethylphenyl)borate, lithium tetrakis(3,5-dimethylphenyl)borate, lithium tetrafluoroborate, sodium tetrakis(pentafluorophenyl)borate, sodium tetraphenylborate, sodium tetrakis(p-tolyl)borate, sodium tetrakis(m-tolyl)borate, sodium tetrakis(2,4-dimethylphenyl)borate, sodium tetrakis(3,5-dimethylphenyl)borate, sodium tetrafluoroborate, potassium tetrakis(pentafluorophenyl)borate, potassium tetraphenylborate, potassium tetrakis(p-tolyl)borate, potassium tetrakis(m-tolyl)borate, potassium tetrakis(2,4-dimethylphenyl)borate, potassium tetrakis(3,5-dimethylphenyl)borate, potassium tetrafluoroborate, lithium tetrakis(pentafluorophenyl)aluminate, lithium tetraphenylaluminate, lithium tetrakis(p-tolyl)aluminate, lithium tetrakis(m-tolyl)aluminate, lithium tetrakis(2,4-dimethylphenyl)aluminate, lithium tetrakis(3,5-dimethylphenyl)aluminate, lithium tetrafluoroaluminate, sodium tetrakis(pentafluorophenyl)aluminate, sodium tetraphenylaluminate, sodium tetrakis(p-tolyl)aluminate, sodium tetrakis(m-tolyl)aluminate, sodium tetrakis(2,4-dimethylphenyl)aluminate, sodium tetrakis(3,5-dimethylphenyl)aluminate, sodium tetrafluoro-aluminate, potassium tetrakis(pentafluorophenyl)aluminate, potassium tetraphenylaluminate, potassium tetrakis(p-tolyl)aluminate, potassium tetrakis(m-tolyl)aluminate, potassium tetrakis(2,4-dimethylphenyl)aluminate, potassium tetrakis (3,5-dimethylphenyl)aluminate, potassium tetrafluoroaluminate, and the like, or combinations thereof. Additional examples and description of ionizing ionic compounds are generally disclosed throughout U.S. Pat. No. 11,186,665.

Chemically treated solid oxides are also suitable activators in the disclosed catalyst compositions. In certain aspects, the chemically treated solid oxides described herein generally can refer to those disclosed, for instance, in U.S. Pat. Nos. 8,536,391 and 10,919,996. In certain aspects, the chemically treated solid oxide can comprise a solid oxide comprising oxygen and at least one element selected from Group 2, 3, 4, 5, 6, 7, 8, 9, 10, 11, 12, 13, 14, or 15 of the periodic table, or comprise oxygen and at least one element selected from the lanthanide or actinide elements; alternatively, the solid oxide can comprise oxygen and at least one element selected from Group 4, 5, 6, 12, 13, or 14 of the periodic table, or comprise oxygen and at least one element selected from the lanthanide elements. (See: Hawley's Condensed Chemical Dictionary, 11th Ed., John Wiley & Sons; 1995; Cotton, F. A.; Wilkinson, G.; Murillo; C. A.; and Bochmann; M. Advanced Inorganic Chemistry, 6th Ed., Wiley-Interscience, 1999.) In some aspects, the inorganic oxide can comprise oxygen and at least one element selected from Al, B, Be, Bi, Cd, Co, Cr, Cu, Fe, Ga, La, Mn, Mo, Ni, Sb, Si, Sn, Sr, Th, Ti, V, W, P, Y, Zn or Zr; alternatively, the inorganic oxide can comprise oxygen and at least one element selected from Al, B, Si, Ti, P, Zn or Zr.

In certain aspects, the chemically treated solid oxide can comprise a solid oxide comprising Al₂O₃, B₂O₃, BeO, Bi₂O₃, CdO, CO₃O₄, Cr₂O₄, CuO, Fe₂O₃, Ga₂O₃, La₂O₃, Mn₂O₃, MoO₃, NiO, P₂O₅, Sb₂O₅, SiO₂, SnO₂, SrO, ThO₂, TiO₂, V₂O₅, WO₃, Y₂O₃, ZnO, ZrO₂, mixed oxides thereof, and combinations thereof. In certain aspects, the solid oxide can comprise silica, alumina, silica-alumina, silica-coated alumina, aluminum phosphate, aluminophosphate, heteropolytungstate, titania, zirconia, magnesia, boria, zinc oxide, a mixed oxide thereof, or any combination thereof. In other aspects, solid oxides can comprise silica-coated alumina.

In certain aspects, the chemically treated solid oxide can comprise a solid oxide treated with at least one electron-withdrawing anion, wherein the solid oxide can comprise any oxide that is characterized by a high surface area, and the electron-withdrawing anion can comprise any anion that increases the acidity of the solid oxide as compared to the solid oxide that is not treated with at least one electron-withdrawing anion.

The solid oxide material can be treated with a source of halide ion, sulfate ion, or a combination thereof, and optionally treated with a metal ion. In one aspect, the solid oxide material can be treated with a source of sulfate (termed a sulfating agent), a source of phosphate (termed a phosphating agent), a source of iodide ion (termed an iodiding agent), a source of bromide ion (termed a bromiding agent), a source of chloride ion (termed a chloriding agent), a source of fluoride ion (termed a fluoriding agent), or any combination thereof, and calcined to provide the chemically treated solid oxide.

In certain aspects, the chemically treated solid oxide can comprise a solid oxide treated with an electron-withdrawing anion, wherein the solid oxide is selected from silica, alumina, silica-alumina, aluminum phosphate, heteropolytungstates, titania, zirconia, magnesia, boria, zinc oxide, mixed oxides thereof, or mixtures thereof, and the electron-withdrawing anion is selected from fluoride, chloride, bromide, phosphate, triflate, bisulfate, sulfate, fluorophosphate, fluorosulfate, or any combination thereof. Thus, in certain aspects, the chemically treated solid oxide can comprise fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, phosphated silica-coated alumina, or any combination thereof. In certain aspects, the chemically treated solid oxide can comprise fluorided alumina, sulfated alumina, fluorided silica-alumina, sulfated silica-alumina, fluorided silica-coated alumina, fluorided-chlorided silica-coated alumina, sulfated silica-coated alumina, or any combination thereof. In other aspects, the chemically treated solid oxide can comprise sulfated alumina and/or fluorided silica-coated alumina.

In certain aspects, chemically treated solid oxides disclosed herein can comprise a calcined solid oxide. Thus, in this aspect, the solid oxide can be calcined or uncalcined; alternatively, calcined; or alternatively, uncalcined. In certain aspects, the solid oxide can be calcined prior to, during, or after the solid oxide compound is contacted with the electron-withdrawing anion source resulting in the chemically treated solid oxide. Calcining of the treated solid oxide is generally conducted in an ambient atmosphere; alternatively, in a dry ambient atmosphere. The solid oxide can be calcined at a temperature from 200° C. to 900° C.; alternatively, from 300° C. to 800° C.; alternatively, from 400° C. to 700° C.; or alternatively, from 350° C. to 550° C. The period of time at which the solid oxide is maintained at the calcining temperature can be 1 minute to 100 hours; alternatively, from 1 hour to 50 hours; alternatively, from 3 hours to 20 hours; or alternatively, from 1 to 10 hours.

Pore characteristics for chemically treated solid oxides of disclosed herein can affect the alpha olefin conversion and oligomer selectivity. In certain aspects, the chemically treated solid oxide can have a pore volume greater than 0.1 mL/g, or greater than 0.5 mL/g. In other aspects, the pore volume can be greater than 0.75 mL/g, or greater than 1 mL/g. In another aspect, the pore volume can be greater than 1.2 mL/g. In yet another aspect, the pore volume can be in a range from 0.5 mL/g to 1.8 mL/g, such as, for example, from 0.8 mL/g to 1.7 mL/g, or from 1 mL/g to 1.6 mL/g.

Chemically treated solid oxides disclosed herein also can be characterized by a surface area in a range from 100 to 1000 m²/g, from 150 to 750 m²/g, or from 200 to 600 m²/g. The surface area of the chemically treated solid oxide can range from 250 to 500 m²/g in another aspect of this invention. Solid oxides having surface areas greater than 300 m²/g, greater than 350 m²/g, greater than 400 m²/g, or greater than 450 m²/g, can be employed in aspects of this invention.

In certain aspects disclosed herein, catalyst composition can further comprise a co-catalyst. In certain aspects, the co-catalyst can comprise an organoaluminum compound, an aluminoxane compound, an organoboron or organoborate compound, an ionizing ionic compound, or a combination thereof; alternatively, the co-catalyst can comprise an organoaluminum compound. In an aspect, suitable organoaluminum compounds can have the formula, (R^{Z})₃Al, wherein each R^{Z} independently can be an aliphatic group having from 1 to 10 carbon atoms. For example, each R^{Z} independently can be methyl, ethyl, propyl, butyl, hexyl, or isobutyl. In another aspect, examples of organoaluminum compounds suitable for use in accordance with the present invention can include, but are not limited to, trialkylaluminum compounds, dialkylaluminum halide compounds, dialkylaluminum hydride compounds, as well as combinations thereof. Specific non-limiting examples of suitable organoaluminum compounds can include trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum (TNOA), and the like, or combinations thereof.

Generally, the organoaluminum compound (or other co-catalyst) can be used in any suitable amount relative to the metallocene compound. In certain aspects, a molar ratio of the co-catalyst to the metallocene compound in the catalyst composition can be in a range from 0.1:1 to 100,000:1, from 1:1 to 10,000:1, from 10:1 to 1,000:1, or from 50:1 to 500:1. Catalyst compositions disclosed herein also may be characterized according to the weight ratio of the metallocene compound to the chemically treated solid oxide, which in certain aspects can be in a range from 1:10 to 1:10,000, from 1:10 to 1:1,000, from 1:10 to 500:1, or from 1:10 to 1:100.

### Oligomerization Processes

In one aspect of this invention, a first oligomerization process can comprise contacting a catalyst composition with a C₄ to C₃₀ alpha olefin monomer and optionally H₂ under oligomerization conditions to produce an oligomer product comprising at least 50 mol % alpha olefin dimer. In this first process, the catalyst composition can comprise a metallocene compound, a chemically treated solid oxide, and a co-catalyst, and the metallocene compound can have formula (I), formula (II), or formula (III) disclosed herein. In another aspect of this invention, a second oligomerization process can comprise contacting a catalyst composition with a C₄ to C₃₀ alpha olefin monomer and optionally H₂ under oligomerization conditions to produce an oligomer product, in which the catalyst composition can comprises a metallocene compound, an activator, and an optional co-catalyst. The activator can comprise an aluminoxane compound, an organoboron or organoborate compound, an ionizing ionic compound, a chemically treated solid oxide, or a combination thereof, and the metallocene compound can have formula (I) or formula (II) disclosed herein.

Oligomerization reactions generally can comprise contacting any catalyst composition described above with an alpha olefin monomer under oligomerization conditions to form an oligomer product. Processes described herein are applicable to a wide range of alpha olefin monomers. In certain aspects, the alpha olefin can comprise, consist essentially of, or consist of, a C₄ to C₃₀ alpha olefin; alternatively, a C₄ to C₁₈ alpha olefin; alternatively, a C₄ to C₁₆ alpha olefin; alternatively, a C₅ to C₁₈ alpha olefin; alternatively, a C₆ to C₁₆ alpha olefin; or alternatively, a C₈ to C₁₂ alpha olefin. In an aspect, the oligomer product can be produced from an alpha olefin comprising, consisting essentially of, or consisting of, a C₆ alpha olefin, a Ca alpha olefin, a C₁₀ alpha olefin, a C₁₂ alpha olefin, a C₁₄ alpha olefin, a C₁₆ alpha olefin, or any combination thereof; alternatively, a C₈ alpha olefin, a C₁₀ alpha olefin, a C₁₂ alpha olefin, or any combination thereof; alternatively, a C₆ alpha olefin; alternatively, a C₈ alpha olefin; alternatively, a C₁₀ alpha olefin; alternatively, a C₁₂ alpha olefin; alternatively, a C₁₄ alpha olefin; alternatively, a C₁₆ alpha olefin; or alternatively, a C₁₈ alpha olefin. The alpha olefin monomer can be linear or branched, and in certain aspects, the alpha olefin monomer can comprise a mixture of alpha olefins, e.g., a mixture of C₈ to C₁₂ alpha olefins, or a mixture of C₁₀ alpha olefins. In one aspect, the alpha olefin can comprise, consist essentially of, or consist of, 1-butene, 1-pentene, 1-hexene, 1-octene, 1-decene, 1-dodecene, 1-tetradecene, 1-hexadecene, or any combination thereof, while in another aspect, the alpha olefin monomer can comprise, consist essentially of, or consist of, 1-hexene; alternatively, 1-octene; alternatively 1-decene; or alternatively, 1-dodecene.

Oligomerization conditions utilized in the oligomerization processes can comprise an oligomerization temperature from −10° C. to 250° C., from 20° C. to 180° C., from 50° C. to 160° C.; alternatively, from 55° C. to 160° C.; alternatively, from 60° C. to 155° C.; alternatively, from 65° C. to 150° C.; alternatively, from 70° C. to 140° C.; or alternatively, from 75° C. to 140° C. In another non-limiting aspect, the oligomerization temperature from can range from 70° C. to 90° C.; alternatively, from 90° C. to 120° C.; or alternatively, from 110° C. to 140° C.

In another non-limiting aspect, the oligomerization conditions utilized in the oligomerization processes disclosed herein can comprise performing the oligomerization reaction in the presence of hydrogen. The hydrogen partial pressure in the oligomerization reaction can be any pressure of hydrogen that does not adversely affect the oligomerization reaction. While not intending to be bound by theory, hydrogen can be used in the oligomerization process to control the oligomer distribution. In some non-limiting aspects, the oligomerization conditions can include a partial pressure of hydrogen at least 0.1 psig and often up to and including a partial pressure of 50 psig. Typical ranges for the hydrogen partial pressure can include from 0.1 psig to 50 psig, from 0.1 psig to 20 psig, from 0.1 psig to 10 psig, from 1 psig to 20 psig, from 1 psig to 10 psig, from 2 psig to 20 psig, or from 2 psig to 10 psig.

Certain ratios of components may be used to control the oligomerization process. For instance, increasing the weight ratio of the metallocene compound in the catalyst composition to the alpha olefin monomer can lead to a higher conversion, but may also lead to a heavier mixture of oligomer products (e.g., less of the desirable dimer and trimer products). Nonetheless, the catalyst composition and alpha olefin monomer can be contacted at a weight ratio of the metallocene compound to the alpha olefin monomer ranging from 1:100 to 1:1,000,000, from 1:1,000 to 1:1,000,000, from 1:1,000 to 1:500,000, or from 1:10,000 to 1:250,000, although not limited thereto.

The activity of the catalyst composition is relatively high. For instance, the activity can be at least 50,000 g oligomer/g metallocene compound per hour (g/g*h), or from 20,000 g/g*h to 180,000 g/g*h, from 40,000 g/g*h to 160,000 g/g*h, or from 60,000 to 120,000 g/g*h, for instance in aspects where the oligomerization conditions comprise an oligomerization temperature of 90° C., and wherein the catalyst composition comprises a TIBA co-catalyst.

The oligomer product often contains a dimer of the alpha olefin monomer, a trimer of the alpha olefin monomer, and higher molecular weight oligomers of the alpha olefin monomer (e.g., tetramers and heavies). Advantageously, the disclosed first and second oligomerization processes can produce oligomer products having a relatively high amount of dimers and trimers that can be useful in subsequent reactions and in the production of polyalphaolefins.

The oligomer product formed by the first and second processes, therefore, can be characterized by the relative amount of specific oligomers. For instance, it can be beneficial to maximize the amount of dimer and trimer, while minimizing heavier oligomers in the oligomer product. Surprisingly, the first and second oligomerization processes are able to operate at high conversions of the alpha olefin monomer without causing a shift in the resulting oligomer product toward heavier oligomers. In certain aspects, the oligomer product can comprise less than or equal to 20 mol %, less than or equal to 15 mol %, less than or equal to 10 mol %, or less than or equal to 5 mol % tetramer. Additionally or alternatively, the oligomer product can comprise at least 60 mol %, at least 70 mol %, at least 80 mol %, at least 85 mol %, at least 90 mol %, or at least 95 mol % of dimer and trimer (total). Unreacted alpha olefin monomer is excluded from the compositional breakdown of the oligomer product.

In certain aspects, and beneficially, the dimer may be the majority component of the oligomer product, and the oligomer product can contain at least 30 mol %, at least 40 mol %, at least 50 mol %, at least 55 mol %, at least 60 mol %, at least 65 mol %, at least 70 mol %, or at least 75 mol % alpha olefin dimer, based on total oligomers in the oligomer product, and excluding unreacted alpha olefin monomer.

Beneficially, the amount of vinylidene produced by the disclosed first and second oligomerization process is relatively high. Often, vinylidene is desirable for its high reactivity relative to internal and branched dimers of the alpha olefin monomer. Also beneficially, this can be accomplished with relatively high conversions of the alpha olefin monomer. Thus, the first and second processes described herein demonstrate excellent conversion of the alpha olefin monomer while maintaining a high dimer and trimer content, and also a high vinylidene content of the dimer in the oligomer product. In certain aspects, the dimer in the oligomer product comprises at least 50 mol %, at least 60 mol %, at least 70 mol %, at least 75 mol %, at least 80 mol %, at least 85 mol %, at least 90 mol %, or at least 95 mol % vinylidene. It follows then, that the amount of internal olefin within the dimer portion of the oligomer product generally can be less than or equal to 15 mol %, less than or equal to 12 mol %, or less than or equal to 10 mol %.

Given that the amount of oligomer product as determined by alpha olefin conversion, and the relative distribution of oligomers within the oligomer product may be dependent on reaction conditions such as temperature, time, and the presence or absence of H₂, direct comparison of the oligomerization processes disclosed herein to conventional processes can illustrate the unexpected effect described directly above. As an example, the disclosed first and second processes can produce an oligomer product comprising less alpha olefin tetramer than that of an otherwise identical process in which the catalyst composition comprises an aluminoxane compound, an organoboron or organoborate compound, or an ionizing ionic compound, instead of the catalyst composition comprising the chemically treated solid oxide and co-catalyst as described herein. As another example, the oligomer product can comprise more alpha olefin dimer, and/or more vinylidene dimer, than that of an otherwise identical process in which the catalyst composition comprises an aluminoxane compound, an organoboron or organoborate compound, or an ionizing ionic compound, instead of the catalyst composition comprising the chemically treated solid oxide and the co-catalyst as described herein.

The first and second oligomerization processes, in certain aspects, can further comprise a step of separating at least a portion of the catalyst composition from the oligomer product using any suitable technique, e.g., by filtration. Likewise, the first and second processes can further comprise a step of separating unreacted alpha olefin monomer from the oligomer product using any suitable technique, e.g., wiped film evaporation, distillation, short path distillation, or any combination thereof. Optionally, the first and second processes can further comprise recycling either or both of the recovered catalyst composition and the recovered unreacted alpha olefin monomer, for instance, for re-use in the first oligomerization process and/or the second oligomerization process.

Further still, the first and second oligomerization processes, in certain aspects, can comprise a step of fractionating the oligomer product into alpha olefin dimer, alpha olefin trimer, and alpha olefin heavies (including alpha olefin tetramer and higher oligomers), using any suitable technique, e.g., wiped film evaporation, distillation, short path distillation, or any combination thereof. Similarly, the first and second processes can further comprise a step of hydrogenating at least a portion of the oligomer product (e.g., alpha olefin trimer) to form a polyalphaolefin. The process of fractionating an oligomer product into several oligomer fractions is generally known and techniques and conditions for carrying out the fractionation, and subsequent separation, purification, and/or hydrogenation steps to transform the oligomer fractions into a polyalphaolefin will be understood by those of skill in the art.

The polyalphaolefin may have certain desirable properties. For example, one desirable property which can be achieved by utilizing a separation step or steps is 100° C. kinematic viscosity. A second desirable property which can be achieved by utilizing a separation step or steps is to achieve a desired flash point. A third desirable property which can be achieved by utilizing a separation step or steps is to achieve a desired fire point. A fourth desirable property which can be achieved by utilizing a separation step or steps is to achieve a desired Noack volatility. A fifth desirable property which can be achieved by utilizing a separation step or steps is to achieve a desired pour point. In an embodiment, the separation step(s) can be utilized to remove lower and/or higher molecular weight oligomers to produce an alpha olefin oligomer product, or an alpha olefin oligomer product which will produce a polyalphaolefin, having a desired 100° C. kinematic viscosity, flash point, fire point, Noack volatility, and/or pour point.

Unexpectedly, the alpha olefin oligomer product as formed by the first and second oligomerization processes employing the catalyst compositions disclosed herein results in exceptional conversion of the alpha olefin and high metallocene activity, but also produces an alpha olefin oligomer product with composition lending to the straightforward preparation of polyalphaolefins with exceptionally low viscosity and volatility. In certain aspects, the polyalphaolefin produced from the oligomer product or any portion thereof, can have a kinematic viscosity at 100° C. of less than or equal to 20 cSt, 10 cSt, 5 cSt, 4 cSt, or 3 cSt.

### EXAMPLES

The disclosure is further illustrated by the following examples, which are not to be construed in any way as imposing limitations to the scope of this disclosure. Various other aspects, modifications, and equivalents thereof, which after reading the description herein, can suggest themselves to one of ordinary skill in the art without departing from the spirit of the present disclosure or the scope of the appended claims.

Gas chromatographic (GC) analyses were performed using a split injection method on a Bruker 430-GC gas chromatograph with a flame ionization detector (FID). Initial oven temperature was 70° C. for 2 minutes and was increased 5° C./min to 290° C. and held for 7 minutes. The column was an all-purpose capillary column (Agilent J&W VF-5 ms, 30 m×0.25 mm×0.25 μm). Data analysis was performed using CompassCDS software.

The distribution of olefin end groups was determined using 1H NMR on a Bruker 300 MHz NMR. Spectra were recorded in CDCl₃ and are reported relative to SiMe₄ as determined by reference to the residual ¹H solvent peak. Integration of the following chemical shift ranges were used to determine the relative amounts of olefin end group: Vinylidene: 4.55-4.75 ppm, Trisubstituted: 4.95-5.15 ppm, Internal: 5.20-5.45 ppm.

The chemically treated solid oxide (CTSO) was a fluorided silica-coated alumina (60:40 alumina:silica by weight) containing 4 wt. % F and having a d50 average particle size of 35 microns, a BET surface area of 450 m²/g, and a pore volume of 1.1 mL/g.

Generally, 1-decene was oligomerized to an oligomer product of dimers, trimers, and tetramers in the presence of one of metallocenes A-F, and the data is shown in Tables I-V below. Metallocenes A-F were prepared as reported in U.S. Pat. No. 11,186,665 and the structures are shown below. Examples 1-6, 12-15, and 20-23 utilized heterogenous metallocene catalyst compositions comprising the metallocene compound and the chemically treated solid oxide (CTSO) noted above, whereas Examples 7-11 and 16-19 were conducted using a metallocene catalyst composition comprising the respective metallocene and either an aluminoxane (MMAO-12) or an organoborate (DTPB, with co-catalyst). Examples 1-11 were conducted in the presence of a H₂ overpressure of 5 psig to the nitrogen atmosphere, whereas Examples 12-23 were conducted in the absence of H₂.

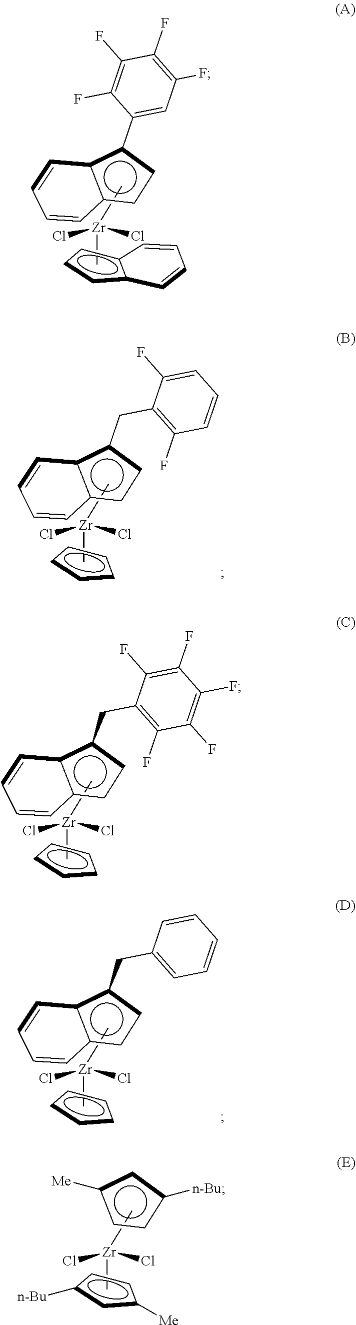

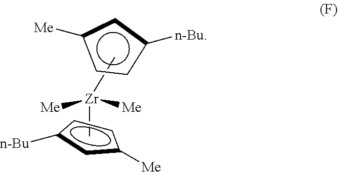

### Metallocene-Catalyzed Oligomerizations of 1-Decene.

Examples 1-6 were prepared as follows. A 1-gallon batch reactor was charged with 1350 g of 1-decene. A syringe was charged with CTSO (1.50 g), TIBA (2.5 mL of a 1.0 M hexane solution), and metallocene (10 mg). The catalyst mixture was shaken to mix and was then charged to the reactor under a nitrogen purge. The reactor was heated to 90° C. (or 110° C. in the case of Example 2 and Example 3-2) while stirring at 600-900 rpm. Once the reactor temperature reached setpoint, hydrogen was charged to the reactor until a pressure of 5 psig was reached. After 1 hour, the reactor was cooled to 35° C. A solution of 10% HCl in isopropyl alcohol (10 mL total charge) was added and the reactor contents were removed.

Examples 7-9 were prepared as follows. A 1-gallon batch reactor was charged with 1350 g of 1-decene. A syringe was charged with metallocene (10 mg) and MMAO-12 (12.0 mL of a 7.0 wt. % toluene solution). The catalyst mixture was shaken to mix and was then charged to the reactor under a nitrogen purge. The reactor was heated to 90° C. while stirring at 600-900 rpm. Once the reactor temperature reached setpoint, hydrogen was charged to the reactor until a pressure of 5 psig was reached. After 1 hour, the reactor was cooled to 35° C. A solution of 10% HCl in isopropyl alcohol (10 mL total charge) was added and the reactor contents were removed.

Examples 10-11 were prepared as follows. A 1-gallon batch reactor was charged with 1350 g of 1-decene and TNOA (0.5 mL of a 25 wt. % hexane solution). A syringe was charged with metallocene (10 mg of metallocene in 1 mL of a 50:50 hexane:toluene mixture). The catalyst mixture was shaken to mix, allowed to stand 5 minutes, and then charged to the reactor under a nitrogen purge. The reactor was heated to 90° C. while stirring at 600-900 rpm. Once the reactor temperature reached setpoint, hydrogen was charged to the reactor until a pressure of 5 psig was reached. After 1 hour, the reactor was cooled to 35° C. A solution of 10% HCl in isopropyl alcohol (10 mL total charge) was added and the reactor contents were removed. The oligomer product was filtered prior to distillation.

Examples 12-19 were performed as follows. A 100 mL round bottomed glass flask equipped with a magnetic stir bar was charged with 50 mL of anhydrous 1-decene and the 1-decene was heated to 90° C. A syringe was charged with the respective catalyst mixture—CTSO (150 mg) where indicated, 3 mL toluene, TIBA (0.5 mL of 1.0 mL hexane solution) or MMAO-12 (0.6 mL of 7 wt. % toluene solution), and 1.5 mg metallocene. The catalyst slurry was charged to the reaction flask and the resulting mixture was stirred for 1 hour. After 1 hour, 1 mL of 10% HCl in isopropyl alcohol was added and the reactor contents were removed. Examples 20-23 were performed similarly, but with the specific conditions shown in Table V.

Following removal from the reactor, oligomer products were filtered through a bed of celite. For Examples 1-11, 200 mL portions were reserved for analysis, while the full reaction mixture (˜50 mL) was utilized for analysis in Examples 12-23. Distillation was performed on all samples to remove unreacted monomer prior to analysis. Samples were first distilled to <7.5 torr at 75° C. and then to <200 mtorr at 55° C.

The composition of oligomerization products of Examples 1-23 was determined by gas chromatography (“GC”) analysis (oligomer distribution) and 1H NMR (olefin distribution) analysis.

### Examples 1-11. Comparison of Different Activators

Table I summarizes Examples 1-6, which each employed a catalyst composition comprising a metallocene catalyst and a CTSO activator. Table II summarizes an analogous set of experiments performed using a catalyst composition comprising either an aluminoxane or organoborate activator. Each of Examples 1-11 was performed with H₂ added during the oligomerization to improve conversion of the alpha olefin monomer.

As shown by the data in Tables I-II, conversion using metallocene F was unexpectedly high and far above that observed for other metallocenes employed. Catalyst compositions comprising metallocenes B and E also demonstrated high conversion in the presence of chemically treated solid oxide and aluminoxane activators, however, metallocene E did not perform well using the organoboron compound DTPB.

Also surprising, Examples 1-6 produced a much lighter oligomer product mixture, as evidenced by the amount of tetramer in the oligomer product being significantly less than that observed in comparable Examples 7-11. Moreover, the vinylidene content observed in Examples 7-11 was generally preserved across Examples 1-6, despite many examples demonstrating a much higher proportion of dimer in the oligomer product mixture. Across Examples 1-11, Metallocene B produced the oligomer product having the lowest tetramer content and highest dimer content, and with excellent conversion (Example 2). Metallocenes E-F with the CTSO activator in Examples 5-6 also resulted in excellent conversion and oligomer product distribution as compared to Examples 9-11.

### Examples 12-23. Comparison of Different Activators without Added H₂

Table III summarizes Examples 12-15, which as for Examples 1-6, employed a catalyst composition comprising a metallocene catalyst and CTSO activator. Table IV summarizes an analogous set of experiments performed using a catalyst composition comprising an aluminoxane activator, similar to Examples 7-11. Each of Examples 12-19 was performed without H₂ added during the oligomerization.

As shown in Tables III and IV, the conversion of alpha olefin monomer decreased significantly compared to Examples 1-11 where H₂ was present. Examples 16-19 had an acceptable conversion and product profile, however, it is clear from comparing the results to Examples 7-11 that increasing the conversion of alpha olefin monomer by adding H₂ does not produce an acceptable oligomer product, as the increase in reactivity also increases the amount of heavies and unreactive dimers in the oligomer product.

Unexpectedly, the same effect is not observed in Examples 12-15. As compared to Examples 12-15, Examples 1-6 had an extraordinary increase in alpha olefin monomer conversion. Surprisingly, the increase in catalyst activity was not accompanied by a dramatic shift in the oligomer product distribution as was seen for Examples 7-11. Particularly, comparing Examples 2 and 13 with Metallocene B, there was only a slight increase in the amount of tetramer with the addition of H₂ (from 3.3 mol % to 5.2 mol %), despite an over 5-fold increase to conversion.

Table V summarizes Examples 20-23, which as for Examples 1-6 and 12-15, employed a catalyst composition comprising a metallocene catalyst and CTSO activator. At the higher oligomerization temperatures, Examples 20-23 had higher conversions and higher catalyst activities as compared to Examples 12-15. Similar to Examples 1-6, Examples 20-23 also produced a light oligomer product mixture (averaging less than 9 mol % tetramer) with high vinylidene content in the dimer fraction (77-78 mol %).

### Oligomer Properties.

Typical properties of C₁₀ dimers and C₁₀ trimers produced as described in the above examples are summarized in Table VI. Also included are typical properties of C₈ dimers and C₈ trimers, produced analogously to the 1-decene experiments described above. For comparison, representative properties of PAO 2 and PAO 4 also are shown.

TABLE I
Summary of results from Examples 1-6.

| | | | | Metallocene | Support | Dimer (mol %) | | | | | |
|---|---|---|---|---|---|---|---|---|---|---|---|
| | | | Conversion | Activity | Activity | | % | % | % | Trimer | Tetramer |
| Ex. | MET | Activator | (wt. %) | (g/g*h) | (g/g*h) | Total | internal | trisubstituted | vinylidene | (mol %) | (mol %) |
| 1 | A | CTSO^{†} | 6.1 | 8184 | 53 | 62.2 | 5.8 | 3.4 | 90.8 | 27.2 | 10.6 |
| 2 | B | CTSO^{†} | 66.8 | 90180 | 579 | 71.9 | 11.9 | 4.8 | 83.3 | 22.9 | 5.2 |
| 3-1* | C | CTSO^{†} | 44.8 | 60490 | 388 | 58.6 | 12.8 | 7.2 | 80.1 | 28.8 | 12.7 |
| 3-2 | C | CTSO^{†} | 58.7 | 79293 | 509 | 62.6 | 14 | 7.8 | 78.2 | 27.4 | 9.9 |
| 4 | D | CTSO^{†} | 19.0 | 25677 | 165 | 57.3 | 8.0 | 5.9 | 86.1 | 31.0 | 11.6 |
| 5 | E | CTSO^{†} | 79.6 | 107483 | 690 | 65.9 | 6.6 | 4.2 | 89.3 | 25.6 | 8.6 |
| 6 | F | CTSO^{‡} | 95.7 | 129195 | 861 | 67.2 | 8.7 | 7.3 | 84.1 | 22.9 | 9.9 |

*Average of two runs;
^{†}TIBA co-catalyst;
^{‡}TNOA co-catalyst;
activity is g oligomer/g metallocene/hr or g oligomer/g CTSO/hr

TABLE II
Summary of results from Examples 7-11.

| | | | | Metallocene | Dimer (mol %) | | | | | |
|---|---|---|---|---|---|---|---|---|---|---|
| | | | Conversion | Activity | | % | % | % | Trimer | Tetramer |
| Ex. | MET | Activator | (wt %) | (g/g*h) | Total | internal | trisubstituted | vinylidene | (mol %) | (mol %) |
| 7* | C | MMAO-12 | 63.5 | 85741 | 39.1 | 11.5 | 6.2 | 82.3 | 35.9 | 25.1 |
| 8 | D | MMAO-12 | 48.1 | 64976 | 36.3 | 8.6 | 3.8 | 87.7 | 49.3 | 14.4 |
| 9 | E | MMAO-12 | 72.9 | 98465 | 43.6 | 3.7 | 2.6 | 93.7 | 37.4 | 19.0 |
| 10 | E | DTPB^{‡} | 2.8 | 3834 | 62.1 | 8.1 | 14.6 | 77.3 | 28.2 | 9.6 |
| 11 | F | DTPB^{‡} | 80.5 | 118800 | 36.2 | 4.3 | 13.3 | 82.4 | 41.6 | 22.2 |

*Average of two runs;
^{‡}TNOA co-catalyst;
DTPB = dimethylanilinium tetrakis(pentafluorophenyl)borate

TABLE III
Summary of results from Examples 12-15.

| | | | | Metallocene | Dimer (mol %) | | | | | |
|---|---|---|---|---|---|---|---|---|---|---|
| | | | Conversion | Activity | | % | % | % | Trimer | Tetramer |
| Ex. | MET | Activator | (wt %) | (g/g*h) | Total | internal | trisubstituted | vinylidene | (mol %) | (mol %) |
| 12 | A | CTSO | 0 | NA | — | — | — | — | — | — |
| 13 | B | CTSO | 11.4 | 2800 | 67.3 | 4.5 | 9.7 | 85.8 | 29.5 | 3.3 |
| 14 | C | CTSO | 4.4 | 1107 | 65.3 | 8.4 | 8.9 | 82.7 | 31.0 | 3.7 |
| 15 | D | CTSO | 0.6 | 150 | 77.5 | 14.2 | 9.8 | 76.0 | 17.3 | 5.1 |

TABLE IV
Summary of results from Examples 16-19.

| | | | | Metallocene | Dimer (mol %) | | | | | |
|---|---|---|---|---|---|---|---|---|---|---|
| | | | Conversion | Activity | | % | % | % | Trimer | Tetramer |
| Ex. | MET | Activator | (wt %) | (g/g*h) | Total | internal | trisubstituted | vinylidene | (mol %) | (mol %) |
| 16 | A | MMAO-12 | 3.6 | 852 | 65.7 | 5.0 | 6.5 | 88.6 | 31.2 | 3.1 |
| 17 | B | MMAO-12 | 73.2 | 17855 | 69.4 | 5.2 | 14.6 | 80.2 | 27.7 | 2.9 |
| 18 | C | MMAO-12 | 29.5 | 7575 | 62.7 | 6.8 | 9.0 | 84.2 | 32.8 | 4.5 |
| 19 | D | MMAO-12 | 31.6 | 8047 | 62.9 | 5.9 | 4.8 | 89.3 | 28.0 | 9.1 |

TABLE V
Summary of results from Examples 20-23.

| | | | | Metallocene | Support | Dimer (mol %) | | | | | |
|---|---|---|---|---|---|---|---|---|---|---|---|
| | | | Conversion | Activity | Activity | | % | % | % | Trimer | Tetramer |
| Ex. | MET | Activator | (wt. %) | (g/g*h) | (g/g*h) | Total | internal | trisubstituted | vinylidene | (mol %) | (mol %) |
| 20 | C | CTSO | 46.4 | 63045 | 420 | 65.6 | 9.7 | 13.2 | 77.1 | 24.7 | 9.7 |
| 21 | C | CTSO | 21.2 | 28811 | 192 | 64.1 | 10.0 | 12.0 | 78.0 | 25.6 | 10.3 |
| 22 | C | CTSO | 22.1 | 30127 | 201 | 67.6 | 9.5 | 12.7 | 77.7 | 23.8 | 8.6 |
| 23 | C | CTSO | 23.1 | 31301 | 209 | 70.8 | 8.5 | 13.9 | 77.6 | 22.3 | 6.9 |

Example 20 - 10 mg metallocene, 1.5 g CTSO, 2.5 mL TIBA (1.0M in hexane), 1350 g 1-decene, 110° C., 60 minutes
Example 21 - 5 mg metallocene, 750 mg CTSO, 1.25 mL TIBA (1.0M in hexane), 675 g 1-decene, 110° C., 60 minutes
Example 22 - 5 mg metallocene, 750 mg CTSO, 1.25 mL TIBA (1.0M in hexane), 675 g 1-decene, 120° C., 60 minutes
Example 23 - 5 mg metallocene, 750 mg CTSO, 1.25 mL TIBA (1.0M in hexane), 675 g 1-decene, 130° C., 60 minutes

TABLE VI
Representative oligomer properties.

| Property | C₈ Dimer | C₈ Trimer | C₁₀ Dimer | C₁₀ Trimer | PAO 2 | PAO 4 |
|---|---|---|---|---|---|---|
| Viscosity (40° C.) | 2.81 | 8.70 | 5.05 | 12.84 | 5.0 | 16.8 |
| Viscosity (100° C.) | 1.17 | 2.45 | 1.81 | 3.30 | 1.7 | 3.85 |
| VI | N/A | N/A | N/A | 130 | N/A | 124 |
| Density at 15° C. (g/mL) | 0.77 | 0.80 | 0.79 | 0.81 | 0.80 | 0.82 |
| Pour Point (° C.) | −44 | −83 | −16 | −68 | −73 | −72 |
| Flash Point (° C.) | 128 | 196 | 160 | N/A | 154 | 217 |
| Noack Volatility (%) | 16.9 (150° C.) | 7.7 (200° C.) | 14.0 (200° C.) | 11.2 | 15.8 (200° C.) | 13.3 |

The disclosure is described above with reference to numerous aspects and specific examples. Many variations will suggest themselves to those skilled in the art in light of the above detailed description. All such obvious variations are within the full intended scope of the appended claims. Other aspects of the disclosure can include, but are not limited to, the following (aspects are described as “comprising” but, alternatively, can “consist essentially of” or “consist of”):
Aspect 1. An oligomerization process comprising:
contacting a catalyst composition with a C₄ to C₃₀ alpha olefin monomer and optionally H₂ under oligomerization conditions to produce an oligomer product comprising at least 50 mol % alpha olefin dimer;
wherein the catalyst composition comprises a metallocene compound, a chemically treated solid oxide, and a co-catalyst; and
wherein the metallocene compound has one of the following formulas:

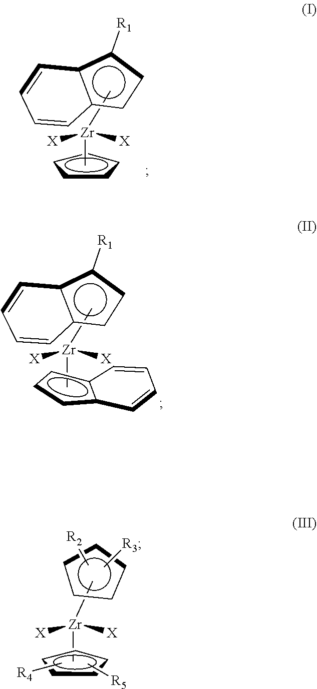

wherein:
R₁ is a C₁-C₂₀ hydrocarbyl group or a halogen-substituted C₁-C₂₀ hydrocarbyl group;
each X independently is a halogen or a C₁-C₁₈ hydrocarbyl group; and
R₂, R₃, R₄, R₅ independently are H or a C₁-C₁₈ hydrocarbyl group.
Aspect 2. An oligomerization process comprising:
contacting a catalyst composition with a C₄ to C₃₀ alpha olefin monomer and optionally H₂ under oligomerization conditions to produce an oligomer product;
wherein the catalyst composition comprises a metallocene compound, an activator, and an optional co-catalyst, wherein the activator comprises an aluminoxane compound, an organoboron or organoborate compound, an ionizing ionic compound, a chemically treated solid oxide, or a combination thereof; and
wherein the metallocene compound has one of the following formulas:

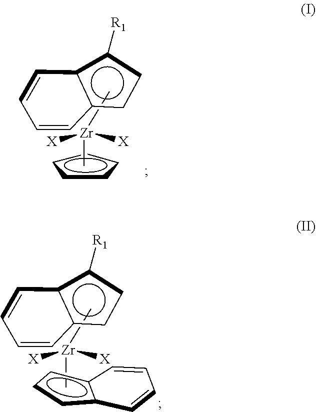

wherein:
R₁ is a halogen-substituted C₁-C₂₀ hydrocarbyl group; and
each X independently is a halogen or a C₁-C₁₈ hydrocarbyl group.
Aspect 3. The process of aspect 1 or 2, wherein the metallocene compound has formula (I) or formula (II), wherein R₁ is a halogen-substituted phenyl group or a halogen-substituted benzyl group.
Aspect 4. The process of any one of aspects 1-3, wherein each X is C₁.
Aspect 5. The process of any one of aspects 1-4, wherein R₁ is a fluorine-substituted C₁-C₂₀ hydrocarbyl group.
Aspect 6. The process of any one of aspects 1-5, wherein R₁ is a fluorophenyl group (e.g., pentafluorophenyl, 2,6-fluorophenyl) or a fluorobenzyl group (e.g., pentafluorobenzyl, 2,4,6-trifluorobenzyl.
Aspect 7. The process of any one of aspects 1-6, wherein the metallocene compound is;

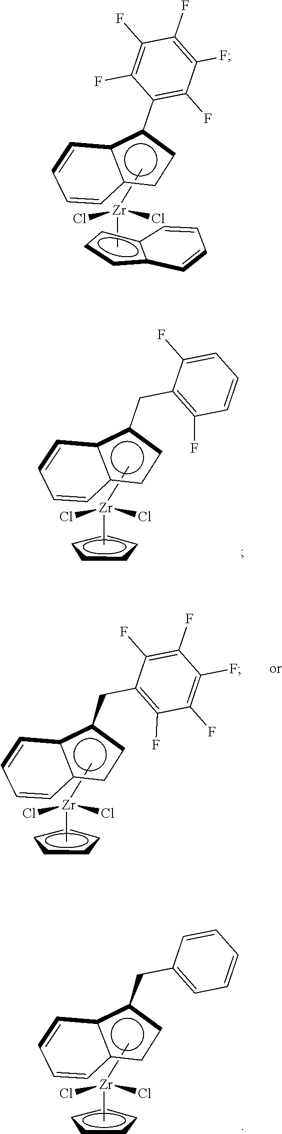

Aspect 8. The process of aspect 1, wherein the metallocene compound is:

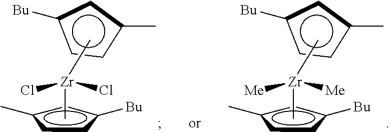

Aspect 9. The process of aspect 1 or 2, wherein the metallocene compound is:

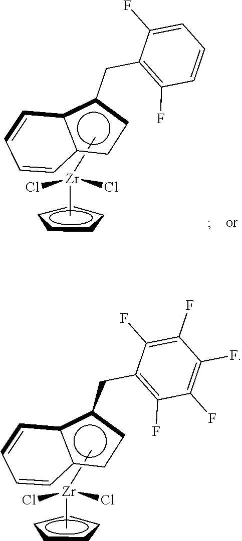

Aspect 10. The process of any one of aspects 1-9, wherein the chemically treated solid oxide comprises fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, phosphated silica-coated alumina, or any combination thereof.
Aspect 11. The process of any one of aspects 1-10, wherein the chemically treated solid oxide comprises fluorided alumina, sulfated alumina, fluorided silica-alumina, sulfated silica-alumina, fluorided silica-coated alumina, fluorided-chlorided silica-coated alumina, sulfated silica-coated alumina, or any combination thereof.
Aspect 12. The process of any one of aspects 1-11, wherein the chemically treated solid oxide comprises a fluorided solid oxide and/or a sulfated solid oxide.
Aspect 13. The process of any one of aspects 1 or 3-12, wherein the co-catalyst comprises an organoaluminum compound, an aluminoxane compound, an organoboron or organoborate compound, an ionizing ionic compound, or a combination thereof.
Aspect 14. The process of any one of aspects 1-12, wherein the co-catalyst comprises an organoaluminum compound.
Aspect 15. The process of aspect 14, wherein the organoaluminum compound comprises trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum (TNOA), or combinations thereof.
Aspect 16. The process of any one of aspects 1-15, wherein a molar ratio of the co-catalyst to the metallocene compound in the catalyst composition is in any range disclosed herein, e.g., from 0.1:1 to 100,000:1, from 1:1 to 10,000:1, from 10:1 to 1,000:1, or from 50:1 to 500:1.
Aspect 17. The process of any one of aspects 1-16, wherein a weight ratio of the metallocene compound to the chemically treated solid oxide is in any range disclosed herein, e.g., from 1:10 to 1:10,000, from 1:10 to 1:1,000, from 1:10 to 500:1, or from 1:10 to 1:100.
Aspect 18. The process of any one of aspects 1-17, wherein the alpha olefin monomer comprises any C₄ to C₁₄ alpha olefin or C₈ to C₁₂ alpha olefin disclosed herein, e.g., 1-octene and/or 1-decene.
Aspect 19. The process of any one of aspects 1-18, wherein the alpha olefin monomer comprises a branched alpha olefin.
Aspect 20. The process of any one of aspects 1-19, wherein the alpha olefin monomer comprises a mixture of alpha olefins (e.g., a mixture of C₈ to C₁₂ alpha olefins, or a mixture of C₁₀ alpha olefins).
Aspect 21. The process of any one of aspects 1-20, wherein a weight ratio of the metallocene compound to the alpha olefin monomer is in any range disclosed herein, e.g., from 1:100 to 1:1,000,000, from 1:1,000 to 1:1,000,000, from 1:1,000 to 1:500,000, or from 1:10,000 to 1:250,000.
Aspect 22. The process of any one of aspects 1-21, wherein the oligomerization conditions comprise an oligomerization temperature in any range disclosed herein, e.g., from −10° C. to 250° C., from 20° C. to 180° C., from 50° C. to 160° C., or from 70° C. to 140° C.
Aspect 23. The process of any one of aspects 1-22, wherein the catalyst composition is contacted with the alpha olefin monomer and H₂ at any suitable hydrogen partial pressure (e.g., from 0.1 to 10 psig of H₂).
Aspect 24. The process of any one of aspects 1-23, wherein an activity of the catalyst composition is in any range disclosed herein, e.g., at least 50,000 g oligomer/g metallocene compound per hour (g/g*h), from 20,000 g/g*h to 180,000 g/g*h, from 40,000 g/g*h to 160,000 g/g*h, or from 60,000 to 120,000 g/g*h under oligomerization conditions comprising an oligomerization temperature of 90° C., and wherein the co-cocatalyst is TIBA.
Aspect 25. The process of any one of aspects 1-24, wherein the oligomer product comprises any amount of tetramer disclosed herein, e.g., less than or equal to 20 mol %, less than or equal to 15 mol %, less than or equal to 10 mol %, or less than or equal to 5 mol %.
Aspect 26. The process of any one of aspects 1-25, wherein the oligomer product comprises less alpha olefin tetramer than that of an otherwise identical process in which the catalyst composition comprises an aluminoxane compound, an organoboron or organoborate compound, or an ionizing ionic compound instead of the chemically treated solid oxide and the co-catalyst.
Aspect 27. The process of any one of aspects 1-26, wherein the oligomer product comprises any amount of alpha olefin dimer and alpha olefin trimer disclosed herein, e.g., at least 60 mol %, at least 70 mol %, at least 80 mol %, at least 85 mol %, at least 90 mol %, or at least 95 mol %.
Aspect 28. The process of any one of aspects 1-27, wherein the oligomer product comprises any amount of alpha olefin dimer disclosed herein, e.g., at least 30 mol %, at least 40 mol %, at least 50 mol %, at least 55 mol %, at least 60 mol %, at least 65 mol %, at least 70 mol %, or at least 75 mol %.
Aspect 29. The process of any one of aspects 1-28, wherein the oligomer product comprises more alpha olefin dimer than that of an otherwise identical process in which the catalyst composition comprises an aluminoxane compound, an organoboron or organoborate compound, or an ionizing ionic compound instead of the chemically treated solid oxide and the co-catalyst.
Aspect 30. The process of any one of aspects 1-29, wherein the alpha olefin dimer comprises any amount of vinylidene disclosed herein, e.g., at least 70 mol %, at least 75 mol %, at least 80 mol %, or at least 85 mol %.
Aspect 31. The process of any one of aspects 1-30, wherein the alpha olefin dimer comprises any amount of internal olefin disclosed herein, e.g., less than or equal to 15 mol %, less than or equal to 12 mol %, or less than or equal to 10 mol %.
Aspect 32. The process of any one of aspects 1-31, wherein the catalyst composition is substantially free of aluminoxane compounds, organoboron or organoborate compounds, ionizing ionic compounds, or combinations thereof.
Aspect 33. The process of any one of aspects 1-32, further comprising a step of separating at least a portion of the catalyst composition from the oligomer product using any technique disclosed herein, e.g., filtration.
Aspect 34. The process of aspect 33, further comprising recycling the separated catalyst composition.
Aspect 35. The process of any one of aspects 1-34, further comprising a step of separating unreacted alpha olefin monomer from the oligomer product using any technique disclosed herein, e.g., wiped film evaporating, distillation, short path distillation, or any combination thereof.
Aspect 36. The process of aspect 35, further comprising recycling unreacted alpha olefin monomer.
Aspect 37. The process of any one of aspects 1-36, further comprising a step of fractionating the oligomer product into alpha olefin dimer, alpha olefin trimer, and alpha olefin heavies including alpha olefin tetramer and higher oligomers, using any technique disclosed herein, e.g., wiped film evaporating, distillation, short path distillation, or any combination thereof.
Aspect 38. The process of any one of aspects 1-37, further comprising a step of hydrogenating at least a portion of the oligomer product (e.g., alpha olefin trimer) to form a polyalphaolefin.
Aspect 39. The process of aspect 38, wherein the polyalphaolefin has a kinematic viscosity at 100° C. of less than or equal to 20 cSt, 10 cSt, 5 cSt, 4 cSt, or 3 cSt (e.g., in a range from 1 to 10 cSt).

We claim:

1. An oligomerization process comprising:
contacting a catalyst composition with a C₄ to C₃₀ alpha olefin monomer and optionally H₂ under oligomerization conditions to produce an oligomer product comprising at least 50 mol % alpha olefin dimer;
wherein the catalyst composition comprises a metallocene compound, a chemically treated solid oxide, and a co-catalyst; and
wherein the metallocene compound has one of the following formulas: 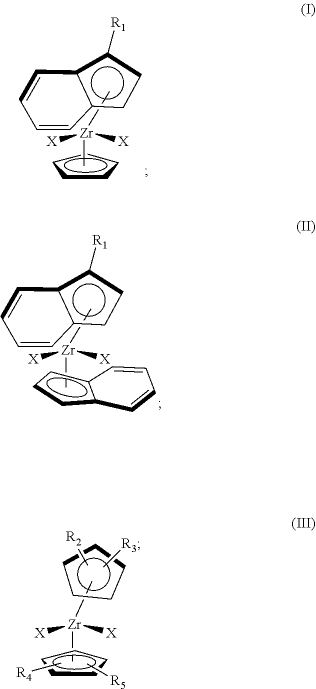
wherein:
R₁ is a C₁-C₂₀ hydrocarbyl group or a halogen-substituted C₁-C₂₀ hydrocarbyl group;
each X independently is a halogen or a C₁-C₁₈ hydrocarbyl group; and
R₂, R₃, R₄, R₅ independently are H or a C₁-C₁₈ hydrocarbyl group.

2. The process of claim 1, wherein R₁ is a fluorophenyl group or a fluorobenzyl group.

3. The process of claim 1, wherein the metallocene compound is:

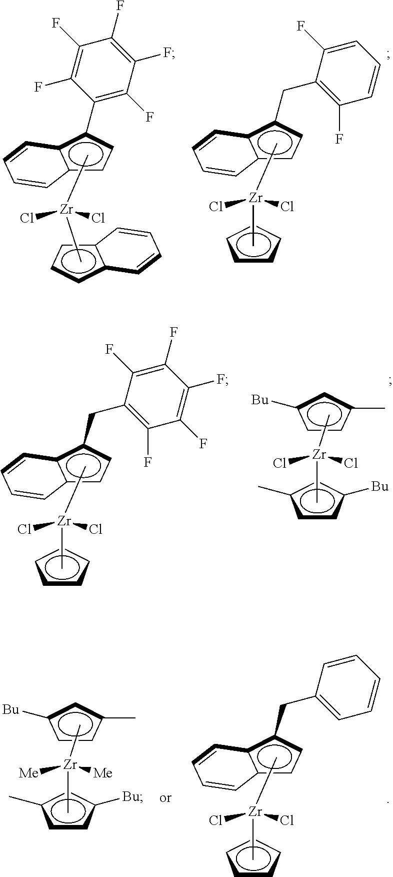

4. The process of claim 1, wherein the metallocene compound is:

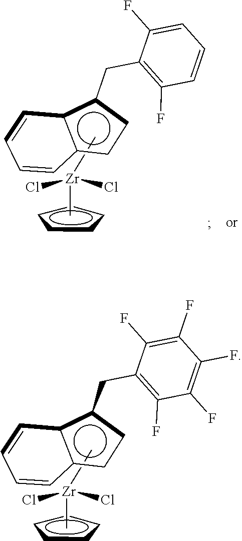

5. The process of claim 1, wherein the chemically treated solid oxide comprises fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, phosphated silica-coated alumina, or any combination thereof.

6. The process of claim 1, wherein the chemically treated solid oxide comprises a fluorided solid oxide and/or a sulfated solid oxide.

7. The process of claim 1, wherein the co-catalyst comprises an organoaluminum compound, an aluminoxane compound, an organoboron or organoborate compound, an ionizing ionic compounds, or a combination thereof.

8. The process of claim 1, wherein the co-catalyst comprises an organoaluminum compound.

9. The process of claim 8, wherein the organoaluminum compound comprises trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum (TNOA), or combinations thereof.

10. The process of claim 1, wherein the alpha olefin monomer comprises a C₄ to C₁₄ alpha olefin.

11. The process of claim 10, wherein the alpha olefin monomer comprises 1-decene.

12. The process of claim 1, wherein a weight ratio of the metallocene compound to the alpha olefin monomer is in a range from 1:10,000 to 1:250,000.

13. The process of claim 1, wherein the catalyst composition is contacted with the alpha olefin monomer and from 0.1 to 10 psig H₂.

14. The process of claim 1, wherein an activity of the catalyst composition is in a range from 20,000 to 180,000 g oligomer/g metallocene/hr under oligomerization conditions comprising an oligomerization temperature of 90° C., and wherein the co-cocatalyst is TIBA.

15. The process of claim 1, wherein:
the oligomer product comprises an amount of alpha olefin dimer of at least 55 mol %; and
the alpha olefin dimer comprises at least 75 mol % vinylidene.

16. The process of claim 1, wherein the oligomer product comprises more alpha olefin dimer than that of an otherwise identical process in which the catalyst composition comprises an aluminoxane compound, an organoboron or organoborate compound, or an ionizing ionic compound instead of the chemically treated solid oxide and the co-catalyst.

17. The process of claim 1, wherein the catalyst composition is substantially free of aluminoxane compounds, organoboron or organoborate compounds, ionizing ionic compounds, or combinations thereof.

18. The process of claim 1, further comprising a step of hydrogenating at least a portion of the oligomer product to form a polyalphaolefin.

19. The process of claim 18, wherein the polyalphaolefin has a kinematic viscosity at 100° C. in a range from 1 to 10 cSt.

20. An oligomerization process comprising:
contacting a catalyst composition with a C₄ to C₃₀ alpha olefin monomer and optionally H₂ under oligomerization conditions to produce an oligomer product;
wherein the catalyst composition comprises a metallocene compound, an activator, and an optional co-catalyst, wherein the activator comprises an aluminoxane compound, an organoboron or organoborate compound, an ionizing ionic compound, a chemically treated solid oxide, or a combination thereof; and
wherein the metallocene compound has one of the following formulas: 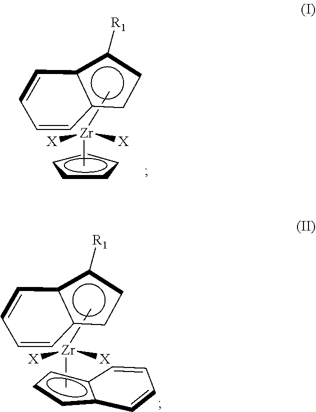
wherein:
R₁ is a halogen-substituted C₁-C₂₀ hydrocarbyl group; and
each X independently is a halogen or a C₁-C₁₈ hydrocarbyl group.
